(19) United States
(12) Patent Application Publication
(10) Pub. No.: US 2016/0113495 A1
(43) Pub. Date: Apr. 28, 2016

(54) ORAL HEALTH MONITORING METHOD AND APPARATUS AND ELECTRONIC DEVICE USING THE SAME

(71) Applicant: SAMSUNG ELECTRONICS CO., LTD., Suwon-si (KR)

(72) Inventors: Ravi NANJUNDAPPA, Bangalore (IN); Rishi FRANKLIN, Bangalore (IN); Amit Vithalrao DHARMAPURIKAR, Bangalore (IN); Seema SINGH, Bangalore (IN)

(73) Assignee: SAMSUNG ELECTRONICS CO., LTD., Suwon-si (KR)

(21) Appl. No.: 14/920,178

(22) Filed: Oct. 22, 2015

(30) Foreign Application Priority Data
Oct. 22, 2014 (IN) 5278/CHE/2014
Sep. 21, 2015 (KR) 10-2015-0133149

(51) Int. Cl. A61B 5/00; A61B 1/00; A61B 1/06; A61B 1/24

(52) U.S. Cl. CPC A61B 5/0013; A61B 1/24; A61B 5/0088; A61B 5/0086; A61B 5/0022; A61B 1/00009; A61B 5/742; A61B 1/063; A61B 1/0646; A61B 5/6898; A61B 2560/0418

(57) ABSTRACT

A health monitoring apparatus including: an optical assembly is configured to: emit a light on a target surface; receive the light reflected from the target surface; collimate the reflected light; and filter the collimated light in a preset wavelength range; and an electronic interface operatively coupled to the optical assembly configured to: trigger the optical assembly; obtain an electrical signal from the optical assembly corresponding to the filtered light; generate data by converting the obtained electrical signals; and transmit the data to an electronic device.

### CROSS-REFERENCE TO RELATED APPLICATION(S)

This application claims priority from Indian Provisional Patent Application No. 5278/CHE/2014 filed on Oct. 22, 2014 in the Indian Patent Office, Indian Patent Application No. 5278/CHE/2014 filed on Aug. 12, 2015 in the Indian Patent Office and Korean Patent Application No. 10-2015-0133149 filed on Sep. 21, 2015 in the Korean Intellectual Property Office, the disclosures of which are incorporated herein by reference in their entireties.

### FIELD

Systems, apparatuses and methods consistent with exemplary embodiments generally relate to the field of oral health care systems, and, more particularly, to an oral health monitoring apparatus and method.

### BACKGROUND

The healthcare domain is becoming smarter and more portable. There are multiple wearable health care devices, which may be in the form of, as a non-limiting example, a wrist watch, like gear fit. The wearable health care devices may connect to a smart phone via an application running on the smartphone and help a user monitor his/her vital signs. Recently, a smartphone case embedded with a plurality of sensors has been developed to monitor heart rate, blood pressure, oxygen level, body temperature etc., of the user. The data can be monitored on a periodic basis and can be availed by the user any-time anywhere. However, the health care domain has not been developed to cover oral monitoring to a greater extent.

Currently available oral care related applications compatible with smartphones provide useful information such as correct brushing pattern in the form of images, home remedies for oral problems, etc. Also, oral monitoring devices are available in the form of mobile phone accessories. For example, an oral cavity scanner (OScan) can be attached to rear camera of a smartphone to detect complete oral cavity. However, applications relating to detection of the most commonly occurring dental issues in real time are not available.

Some of the most commonly occurring dental issues are dental cavity and plaque. Dental cavities are caused due to the presence of cavity causing bacteria, S. Mutans. In a conventional method, dentists use a laser based equipment (called Diagnodent) to detect the dental issues. The Diagnodent includes a laser and photo-sensor embedded in a stylus, which acts as an emitter and a sensor of light, and can be used as a tool for examining the above commonly faced dental issues. Yet, the related device can only be handled by trained professionals, such as dentists.

Hence, there is a need for compact tool or equipment for monitoring oral health system for common/regular usage by smartphone users.

### SUMMARY

According to an aspect of an exemplary embodiment, there is provided a health monitoring apparatus including: an optical assembly is configured to: emit a light on a target surface; receive the light reflected from the target surface; collimate the reflected light; and filter the collimated light in a preset wavelength range; and an electronic interface operatively coupled to the optical assembly configured to: trigger the optical assembly; obtain an electrical signal from the optical assembly corresponding to the filtered light; generate data by converting the obtained electrical signals; and transmit the data to an electronic device.

The optical assembly may be further configured to emit a laser light in a wavelength ranging from 650 to 655 nanometers (nm).

The optical assembly may be configured to filter the collimated light in the wavelength ranging from 680 to 800 nanometers (nm).

The health monitoring apparatus may be formed as a stylus pen.

The health monitoring apparatus is detachably connected to the electronic device.

The optical assembly may include a photodiode.

The photodiode may be configured to detect light having wavelength more than 680 nm.

The electronic interface may be configured to perform at least one of a near field communication (NFC), and Bluetooth low energy (BLE) communication with the electronic device.

The photodiode may be configured to detect light and output an electric signal, and the electronic interface may include: an amplifier configured to amplify the electrical signal obtained from the photodiode; a microcontroller, operatively coupled to amplifier, configured to convert output values received from the amplifier into a digital data; and a power source configured to provide power to the electronic interface.

The health monitoring apparatus may be configured to detect data on corresponding to dental cavities and dental plaque.

The health monitoring apparatus may further include a triggering mechanism configured to determine an activation mode of the health monitoring apparatus.

The electronic interface may be further configured to, in response to the triggering mechanism being activated, transmit a reference signal to the electronic device to activate a corresponding dental application running in the electronic device

The health monitoring apparatus may further include a camera configured to capture images of the target surface.

According to an aspect of another exemplary embodiment, there is provided an electronic device in a wireless communication system, including: a processor; a transceiver, operatively coupled to the processor, configured to receive a data from a health monitoring apparatus; and a display operatively coupled to the processor; wherein the processor is configured to control the display to display the data, wherein the health monitoring apparatus includes an optical assembly and an electronic interface, wherein the optical assembly is configured to: emit a light on a target surface; receive the light reflected from the target surface; collimate the reflected light; and filter the collimated light in a preset wavelength range, and wherein the electronic interface is configured to: trigger the optical assembly; obtain an electrical signal from the optical assembly corresponding to the filtered light; generate the data by converting the obtained electrical signals; and transmit the data to the electronic device.

The data may be related to one or more oral conditions.

The transceiver may be further configured to receive a reference signal from the health monitoring apparatus, and the processor may be further configured to activate a dental application in the electronic device in response to the transceiver receiving the reference signal

The transceiver may be further configured to transmit the data to a server.

According to an aspect of another exemplary embodiment, there is provided a method for a health monitoring apparatus in a health monitoring system, the method including: emitting a light on a target surface; receiving the light reflected from the target surface; collimating the received light; filtering the collimated light in a preset wavelength; obtaining an electrical signal corresponding to the filtered collimated light from an optical assembly; generating data by converting the obtained electrical signal; and transmitting the data to an electronic device.

According to an aspect of another exemplary embodiment, there is provided a method for an electronic device in a wireless communication system, the method including: receiving a data from a health monitoring; and displaying the data on a display of the electronic device, wherein the data is generated by converting an electronic signal, wherein the electronic signal is obtained from an optical assembly by using a light, and wherein the optical assembly is configured to: emit the light on a target surface; receive the light reflected from the target surface; and collimate the received light.

The method may further include transmitting the data to a server.

According to an aspect of another exemplary embodiment, there is provided an oral health monitoring apparatus including: a laser diode configured to emit light towards a tooth surface; an optical sensor configured to detect the light reflected from the tooth surface; a collimator lens configured to collimate the detected light; a filter configured to filter the collimated light; a photo diode configured to receive the collimated light and to output an analog electronic signal corresponding to the collimated light; a microcontroller configured to convert the analog electronic signal to digital electronic data; and a transceiver configured to transmit the digital electronic data to an external device.

The digital electronic data may correspond to an oral condition of the tooth.

The oral condition may correspond to at least one of a cavity state and plaque state of the tooth.

### BRIEF DESCRIPTION OF THE DRAWINGS

The aforementioned aspects and other features of one or more exemplary embodiments will be explained in the following description, taken in conjunction with the accompanying drawings, wherein:

FIG. 1 is a block diagram of an oral health monitoring apparatus, according to an exemplary embodiment.

FIG. 2 is a schematic diagram illustrating communication between communication devices and oral product companies over a cloud server for sharing data related to oral conditions, according to an exemplary embodiment.

FIG. 3 is a schematic diagram illustrating communication between communication device and different entities over a cloud server for sharing data related to one or more oral conditions, according to another exemplary embodiment.

FIG. 4 is a schematic diagram illustrating operating modes of an apparatus, according to an exemplary embodiment.

FIG. 5 is a schematic diagram illustrating exemplary output data corresponding to an oral condition detected by an oral monitoring apparatus on a display of a communication device, according to an exemplary embodiment.

Although specific features of one or more exemplary embodiments are shown in some drawings and not in others, this is done for convenience only, as each feature may be combined with any or all of the other features in accordance with one or more exemplary embodiments.

### DETAILED DESCRIPTION

One or more exemplary embodiments relate an apparatus, an electronic device and a method for monitoring health system. The electronic device may be a communication device. The health system may be an oral health system. One or more exemplary embodiments will now be described in detail with reference to the accompanying drawings. However, the described exemplary embodiments are non-limiting. One or more exemplary embodiments can be modified in various forms. Thus, one or more exemplary embodiments are only provided to explain more clearly aspects of the present invention to one of ordinary skilled in the art. In the accompanying drawings, like reference numerals are used to indicate like components.

The specification may refer to “an,” “one,” or “some” exemplary embodiment(s) in several locations. This does not necessarily imply that each such reference is to the same exemplary embodiment(s), or that the feature only applies to a single exemplary embodiment. Single features of different exemplary embodiments may also be combined to provide other exemplary embodiments. As used herein, the singular forms “a,” “an,” and “the” are intended to include the plural forms as well, unless expressly stated otherwise. It will be further understood that the terms “includes,” “comprises,” “including,” and/or “comprising” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations and arrangements of one or more of the associated listed items.

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

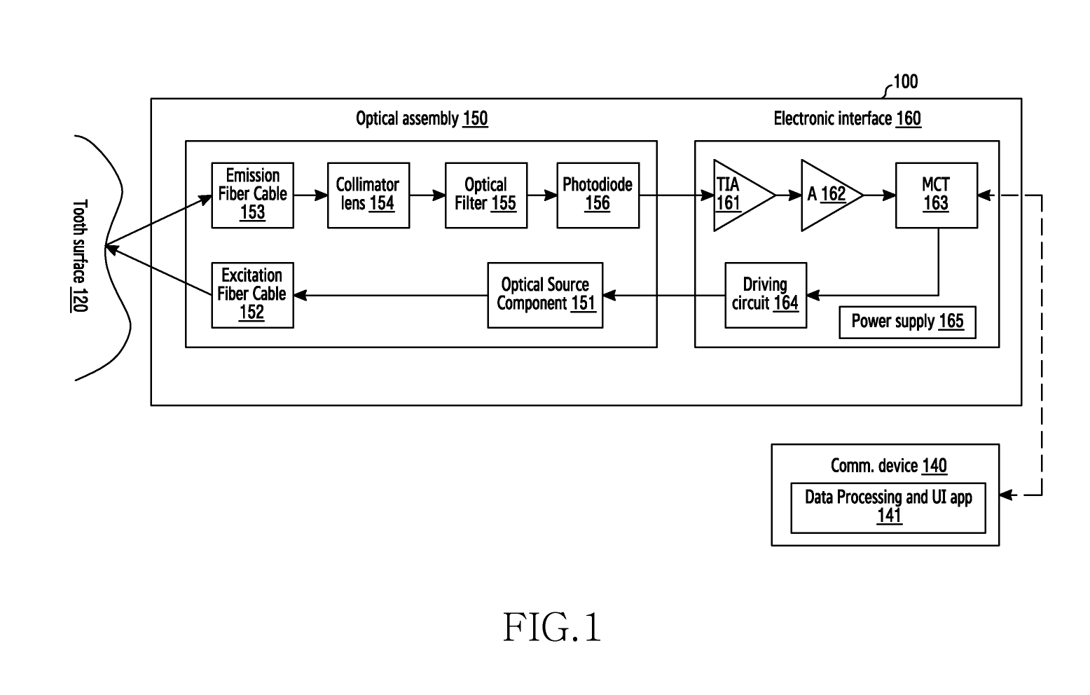

FIG. 1 is a block diagram of an oral health monitoring apparatus according to an exemplary embodiment. As shown in FIG. 1, the apparatus 100 consists of an optical assembly 150, e.g., an optical assembly unit, and an electronic interface 160, e.g., an electronic interface unit, for monitoring oral health. The optical assembly 150 includes an optical source component 151, an excitation fiber cable 152, an emission fiber cable 153, e.g., an optical sensor, a collimator lens 154, an optical filter 155, and a photodiode 156. The electronic interface 160 includes a trans-impedance amplifier 161, a non-inverting operational amplifier 162, a microcontroller 163, a driving circuit 164, and a power supply 165.

The optical source component 151 is adapted for emitting light energy upon receiving a trigger from a driving circuit 164. In an exemplary embodiment, the optical source component 151 includes a laser diode for emitting laser light on a target (tooth) surface 120. Unlike other diodes, the laser diode is more capable for triggering emissions in cavity and plaque bacteria. The clinical studies reveals that an X-ray based cavity detection technique provides 57% accuracy in detecting oral conditions, whereas the laser based cavity detection technique provides results with 90% accuracy. In an exemplary embodiment, the laser diode is adapted to emit laser light of wavelength used in dentistry 650-655 nm on a tooth surface 120 using the excitation fiber cable 152. The reflections from the tooth surface 120 are received via the emission fiber cable 153.

The wavelength of the reflected laser light ranges from 680-800 nm. The reflected laser light is deviated when it is received via the emission fiber cable 153. The deviations are due to numerical aperture of the fiber and minor imperfections caused during fiber manufacturing. Further, the reflected wavelength also contains some wavelengths of laser light associated with the optical source component 151. The collimator lens 154 is used to collimate the scattered emission light received from the emission fiber cable 153. The collimated light is then made to pass through the optical filter 155. The optical filter 155 mat include a long pass filter. The long pass filter has a capability of allowing light rays which are perpendicular to the surface to pass through the filter. So, when the collimated laser light passes through the long pass filter, the long pass filter filters only the required spectrum ranging from 680-800 nm wavelength. The output of long pass filter is again fed into the photodiode 156, where the photodiode 156 detects wavelengths greater than 680 nm. The electrical signal obtained from the photodiode 156 is then fed into the electronic interface 160.

The electronic interface 160 receives electrical signal from the photodiode 156 of the optical assembly 150. The electronic interface 160 is adapted to convert analog values into digital values. To do so, the analog signals are amplified using amplifiers, namely the trans-impedance amplifier 161 and the non-inverting operational amplifier 162. The amplified signal is then fed into the microcontroller 163. The microcontroller 163 includes an internal analog to digital converter (ADC) for converting analog values obtained from the optical assembly 150 into digital values. The digital values are then transferred via an interface, i.e., a transceiver, to a communication device 140 for further processing by a dental application running in the communication device 140.

The electronic interface 160 further includes a driving circuit 164 and a power supply 165. The driving circuit 164 is adapted to drive the optical source component 151 present in the optical assembly 150 to emit the light energy and the power supply 165 is adapted to provide power to one or more components of the oral health monitoring apparatus 100.

In one exemplary operation, the optical source component 151 includes a laser diode. Accordingly, a laser diode driving circuit is used in electronic interface circuit 160 for triggering the laser diode present in the optical source component 151. The laser diode driving circuit triggers the laser diode to emit laser light in the range of wavelength 650-655 nm on the tooth surface 120 via the excitation fiber cable 152. The power to the laser diode driving circuit is provided by the microcontroller 163. The power supply 165 also provides power to one or more components of the oral health monitoring apparatus 100. In an exemplary embodiment, lithium polymer (LiPo) batteries are used as a power source of the power supply 165 for triggering the driving circuit 164, photodiode 156 etc.

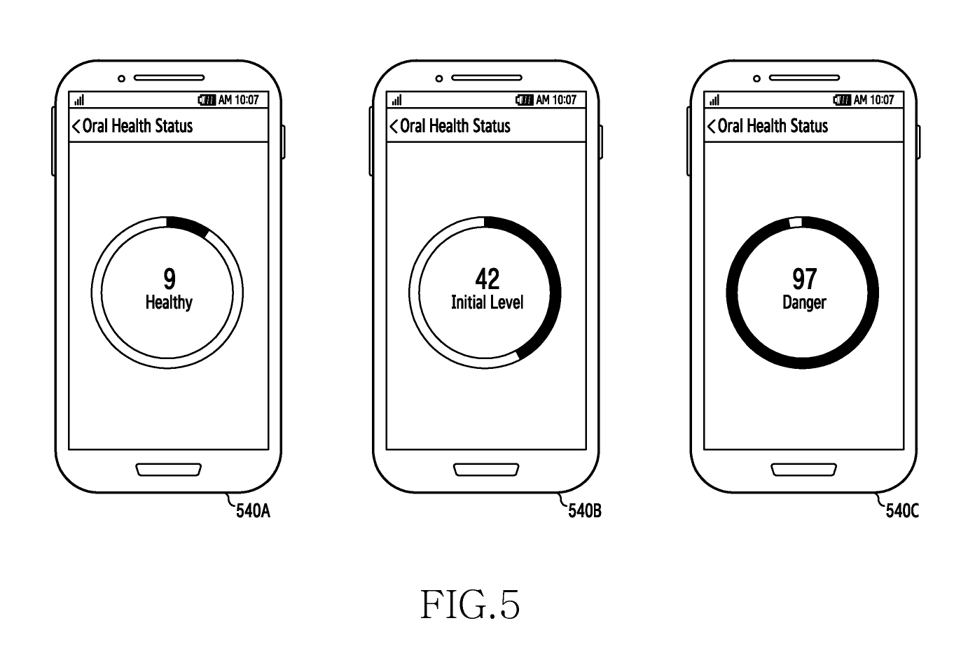

The microcontroller 163 of the electronic interface 160 includes an interface for communicating the digital values to the communication device 140. The interface may include at least one of a near field communication (NFC) and a Bluetooth low energy (BLE) communication. A data processing and user interface dental application residing in the communication device 140 receives the digital values, and further processes the digital values to provide output values relating to one or more oral conditions on a display of the communication device 140. Exemplary output data indicating one or more oral conditions are discussed below with reference to FIG. 5. The one or more oral conditions include at least one of dental cavities and plaque. The communication device 140 may be a mobile phone, smart phone, personal digital assistant, tablet, phablet, laptop, and the like.

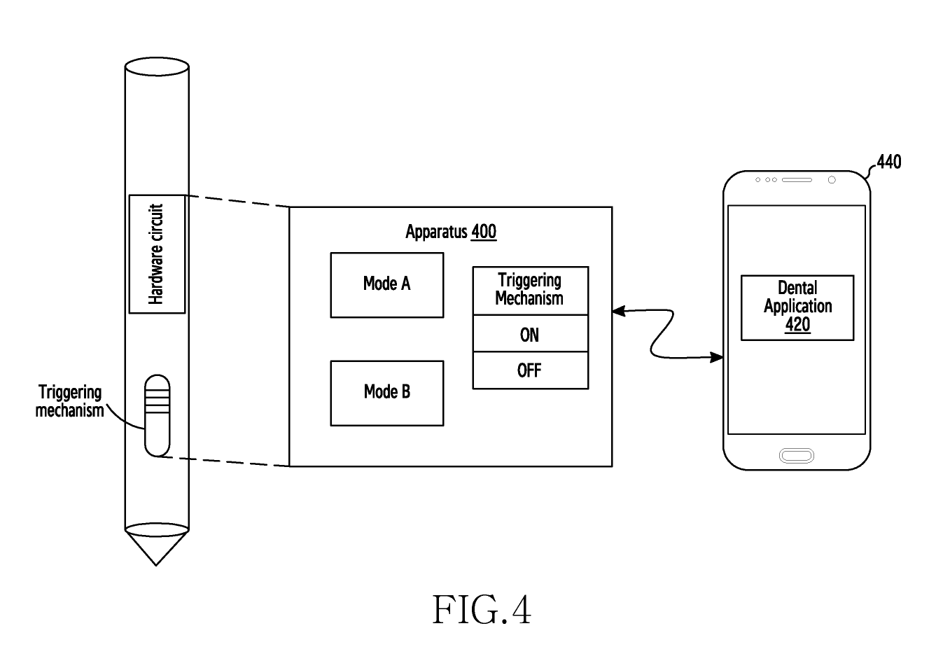

In an exemplary embodiment, the apparatus 100 may have a camera sensor to capture the view of inside mouth while scanning for cavities. If cavities are present, then an image of those teeth are highlighted in different color and displayed on the display of the communication device 140. In some exemplary embodiments, the apparatus is detachably connected to the communication device 140. In these exemplary embodiments, the apparatus acts like a stylus-pen and the stylus pen resides on a side of the communication device 140. The apparatus 100 includes a triggering mechanism for enabling/disabling the function of the stylus to detect one or more oral conditions. When the triggering mechanism is ON, the apparatus is able to detect one or more oral conditions. The triggering mechanism includes at least one of a press type-button, and a slide type button. Thus, the apparatus 100 operates in two modes namely mode A or mode B as shown in FIG. 4. The apparatus may also include an alarm circuit which alerts a user with a beep sound after scanning/screening of mouth is completed. Thus, the user is notified of time period, the apparatus 100 needs to be kept inside the mouth.

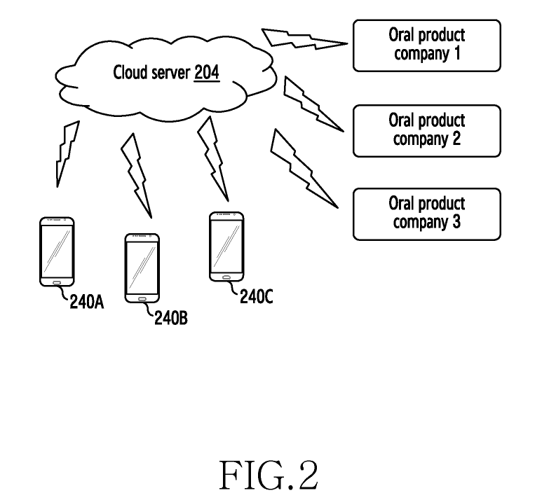

FIG. 2 is a schematic diagram illustrating communication between communication devices and oral product companies over a cloud server for sharing data related to oral conditions, according to an exemplary embodiment. In one or more exemplary embodiments, the apparatus uses wireless communication, such as near field communication (NFC) and Bluetooth low energy communication (BLE), to communicate with the communication device. As shown in FIG. 2, the communication devices 240A, 240B, and 240C store the output values relating to one or more oral conditions in a cloud server 204. The data can be stored on, as non-limiting examples, a weekly or monthly basis in the cloud server 204. In one or more exemplary embodiments, the data can be shared with one or more oral product companies. The one or more oral product companies can access the data stored in the cloud server for the betterment of their oral products.

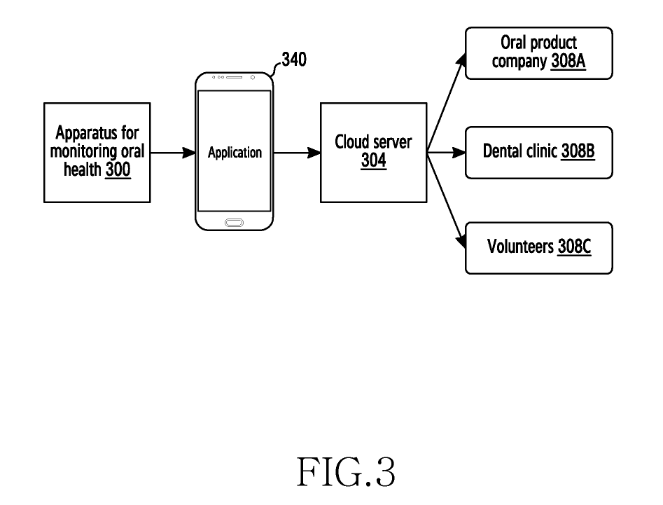

In one or more exemplary embodiments, the data stored in the cloud server can be provided to different entities. FIG. 3 is a schematic diagram illustrating communication between communication device and different entities over a cloud server for sharing data related to one or more oral conditions determined by an apparatus, according to an exemplary embodiment. As shown in FIG. 3, the apparatus 300 determines one or more oral conditions and corresponding values relating to one or more oral conditions are sent to the communication device 340. An application running in the communication device 340 further process the values and store the data in a cloud server 304. By storing the data relating to oral conditions on the cloud server, the user can keep a track of his oral conditions for future analysis. In one or more exemplary embodiments, the user is able to share the oral health data with different kinds of entities. For example, the data can be shared with an oral product manufacturing company 308A, where the company is able to analyze a quality of a product and the capability of the product to cure one or more conditions. The user can share the oral health report to a dental clinic 308B where the user undergoes regular checkups, so that the user can be alerted for treatment/surgeries based on his oral health report. A user may set preferences to share oral data with third party entities, such as volunteers 308C who use the oral data to conduct surveys or studies. The volunteers 308C can access the cloud server 304 to take the readings associated with oral conditions associated with people in that particular region.

FIG. 4 is a schematic diagram illustrating operating modes of an apparatus, according to one or more exemplary embodiments. As shown in FIG. 4, the apparatus 400 operates in two modes namely mode A or mode B. The modes of operation of the apparatus can be changed based on a triggering mechanism. When the triggering mechanism is ON, mode A will be activated and, when the triggering mechanism is OFF, mode B will be activated. The triggering mechanism may include at least one of a press type button, a slide button etc. When mode A is activated, a dental application 420 residing in the communication device 440 also is activated. Later on, one or more oral conditions detected by the apparatus 400 are converted into digital values and transmitted wirelessly to the communication device 440. The dental application 420 further processes the obtained data and displays the result on a display of the communication device 440.

FIG. 5 is a schematic diagram illustrating an exemplary output data corresponding to an oral condition detected by an oral health monitoring apparatus on a display of a communication device, according to one or more exemplary embodiments. Values obtained from the oral health monitoring apparatus are processed further by a dental application running on the communication device and the data relating to one or more oral conditions are displayed on the display of the communication device in a user friendly manner. As non-limiting examples, the output values may include numerical values and pictorial representation indicating a severity associated with the oral condition, wherein the numerical value ranges from 1-100. As shown in FIG. 5, the output values indicate severity of the oral condition whether it is healthy (540A), at initial level (540B), or at a dangerous level (540C). These values can be stored in the dental application and can be shared in a cloud server for future reference.

According to an exemplary embodiment, the electronic device includes a processor, a transceiver operatively coupled to the processor, and a display operatively coupled to the processor. The transceiver is configured to receive a data obtained from an apparatus. The processor is configured to display the obtained data on the display.

According to an exemplary embodiment, the data may be a data related to one or more oral conditions.

According to an exemplary embodiment, the transceiver may receive a reference signal for the triggering mechanism. The transceiver also may activate a dental application in the electronic device in response to the reference signal.

According to an exemplary embodiment, the electronic device is further configured to transmit the data to a server. The server may be a cloud server.

According to an exemplary embodiment, a method for an apparatus in a health monitoring system include emitting a light on a target surface, receiving the light reflected from the target surface, collimating the received light, filtering the collimated light having a preset wavelength, obtaining an electrical signal by using the filtered collimated light from an optical assembly, generating a data by converting the obtained electrical signal, and transmitting the data to the electronic device.

According to an exemplary embodiment, a method for an electronic device in a wireless communication system includes receiving a data from at least one apparatus, and displaying the data on a display of the electronic device. The data is generated by converting an electronic signal. The electronic signal is obtained from an optical assembly by using a light. The optical assembly is configured to emit the light on a target surface. The optical assembly is also configured to receive the light reflected from the target surface. The optical assembly is also configured to collimate the received light.

One or more exemplary embodiments have been described with reference to specific exemplary embodiments; it will be evident that various modifications and changes may be made to these exemplary embodiments without departing from the broader spirit and scope of the various exemplary embodiments. Although various specific exemplary embodiments are described herein, it will be obvious for a person skilled in the art to practice the invention with modifications. However, all such modifications are deemed to be within the scope of the claims and their equivalents. It is also to be understood that the following claims are intended to cover all of the generic and specific features of various exemplary embodiments described herein and their equivalents, and all the statements of the scope of the exemplary embodiments which as a matter of language might be said to fall there between.

What is claimed is:

1. A health monitoring apparatus comprising:
an optical assembly is configured to:
emit a light on a target surface,
receive the light reflected from the target surface,
collimate the reflected light, and
filter the collimated light in a preset wavelength range; and
an electronic interface operatively coupled to the optical assembly configured to:
trigger the optical assembly,
obtain an electrical signal from the optical assembly corresponding to the filtered light,
generate data by converting the obtained electrical signals, and
transmit the data to an electronic device.

2. The health monitoring apparatus as claimed in claim 1, wherein the optical assembly is further configured to emit a laser light in a wavelength ranging from 650 to 655 nanometers.

3. The health monitoring apparatus as claimed in claim 1, wherein the optical assembly is configured to filter the collimated light in the wavelength ranging from 680 to 800 nanometers.

4. The health monitoring apparatus as claimed in claim 1, wherein the health monitoring apparatus is formed as a stylus pen.

5. The health monitoring apparatus as claimed in claim 1, wherein the health monitoring apparatus is detachably connected to the electronic device.

6. The health monitoring apparatus as claimed in claim 1, wherein the optical assembly further comprises a photodiode.

7. The health monitoring apparatus of claimed in claim 6, wherein the photodiode is configured to detect light having wavelength more than 680 nm.

8. The health monitoring apparatus as claimed in claim 1, wherein the electronic interface is configured to perform at least one of a near field communication, and Bluetooth low energy communication with the electronic device.

9. The health monitoring apparatus as claimed in claim 6, wherein the photodiode is configured to detect light and output an electric signal, and
the electronic interface comprises:
an amplifier configured to amplify the electrical signal obtained from the photodiode;
a microcontroller, operatively coupled to amplifier, configured to convert output values received from the amplifier into a digital data; and
a power source configured to provide power to the electronic interface.

10. The health monitoring apparatus as claimed in claim 1, wherein the health monitoring apparatus is configured to detect data corresponding to dental cavities and dental plaque.

11. The health monitoring apparatus as claimed in claim 1, further comprising a triggering mechanism configured to determine an activation mode of the health monitoring apparatus.

12. The health monitoring apparatus as claimed in claim 11, wherein the electronic interface is further configured to, in response to the triggering mechanism being activated, transmit a reference signal to the electronic device to activate a corresponding dental application running in the electronic device

13. The health monitoring apparatus as claimed in claim 1, further comprising a camera configured to capture images of the target surface.

14. An electronic device in a wireless communication system, the electronic device comprising:
a processor;
a transceiver, operatively coupled to the processor, configured to receive a data from a health monitoring apparatus; and
a display operatively coupled to the processor;
wherein the processor is configured to control the display to display the data,
wherein the health monitoring apparatus comprises an optical assembly and an electronic interface,
wherein the optical assembly is configured to:
emit a light on a target surface,
receive the light reflected from the target surface,
collimate the reflected light, and
filter the collimated light in a preset wavelength range, and wherein the electronic interface is configured to:
trigger the optical assembly,
obtain an electrical signal from the optical assembly corresponding to the filtered light,
generate the data by converting the obtained electrical signals, and
transmit the data to the electronic device.

15. The electronic device as claimed in claim 14, the data is related to one or more oral conditions.

16. The electronic device as claimed in claim 14, wherein the transceiver is further configured to receive a reference signal from the health monitoring apparatus, and
the processor is further configured to activate a dental application in the electronic device in response to the transceiver receiving the reference signal

17. The electronic device as claimed in claim 14, wherein the transceiver is further configured to transmit the data to a server.

18. A method for a health monitoring apparatus in a health monitoring system, the method comprising:
emitting a light on a target surface;
receiving the light reflected from the target surface;
collimating the received light;
filtering the collimated light in a preset wavelength;
obtaining an electrical signal corresponding to the filtered collimated light from an optical assembly;
generating data by converting the obtained electrical signal; and
transmitting the data to an electronic device.

19. A method for an electronic device in a wireless communication system, the method comprising:
receiving a data from a health monitoring; and
displaying the data on a display of the electronic device,
wherein the data is generated by converting an electronic signal,
wherein the electronic signal is obtained from an optical assembly by using a light, and
wherein the optical assembly is configured to:
emit the light on a target surface,
receive the light reflected from the target surface, and
collimate the received light.

20. The method as claimed in claim 19, further comprising transmitting the data to a server.

21. An oral health monitoring apparatus comprising:
a laser diode configured to emit light towards a tooth surface;
an optical sensor configured to detect the light reflected from the tooth surface;
a collimator lens configured to collimate the detected light;
a filter configured to filter the collimated light;
a photo diode configured to receive the collimated light and to output an analog electronic signal corresponding to the collimated light;
a microcontroller configured to convert the analog electronic signal to digital electronic data; and
a transceiver configured to transmit the digital electronic data to an external device.

22. The oral health monitoring apparatus as claimed in claim 21, wherein the digital electronic data corresponds to an oral condition of the tooth.

23. The oral health monitoring apparatus as claimed in claim 22, wherein the oral condition corresponds to at least one of a cavity state and plaque state of the tooth.
